(19) United States
(12) Patent Application Publication
(10) Pub. No.: US 2020/0288963 A1
(43) Pub. Date: Sep. 17, 2020

(54) ARTIFICIAL INTELLIGENCE EYE DISEASE SCREENING SERVICE METHOD AND SYSTEM

(71) Applicant: Zhongshan Ophthalmic Center of Sun Yat-sen University, Guangdong (CN)

(72) Inventors: Haotian LIN, Guangdong (CN); Xiaohang WU, Guangdong (CN); Weiyi LAI, Guangdong (CN)

(21) Appl. No.: 16/535,804

(22) Filed: Aug. 8, 2019

(30) Foreign Application Priority Data
Mar. 12, 2019 (CN) 201910184885.8

(51) Int. Cl. A61B 3/00; G16H 15/00; G16H 50/20; G16H 30/20

(52) U.S. Cl. CPC A61B 3/0025; G16H 15/00; A61B 3/028; G16H 30/20; G16H 50/20

(57) ABSTRACT

The invention relates to an artificial intelligence eye disease screening service method and a system, in which the method includes acquiring the ophthalmic examination data of a user, in which the ophthalmic examination data is obtained by a technician in a primary hospital through an ophthalmic examination on the user; performing the artificial intelligence screening on the ophthalmic examination data to obtain a screening result; determining whether the screening result is normal or not; generating an ophthalmic examination report if the screening result is normal; and uploading the ophthalmic examination data to ophthalmologists of the superior hospitals for review and generating an ophthalmic examination report according to the review if the screening result is abnormal. The invention can partly balance the ophthalmic resources between collaborative primary hospitals and superior hospitals, and achieve a large-scale and highly efficient eye disease screening with wide coverage.

### TECHNICAL FIELD

The invention relates to the technical field of artificial intelligence medical treatment, and more particularly relates to an artificial intelligence eye disease screening service method and a system.

### BACKGROUND ART

The imbalance between supply and demand in the medical system, which manifest as expensive medical bills and the difficulty of getting medical services, is a prevalent problem. The scarcity and unbalanced distribution of the medical resources, and a low efficiency make it impossible to meet the rapidly growing medical needs of the people.

In particular to the ophthalmological field, the screening of common eye diseases primarily relies on manual services, while high-quality ophthalmic medical resources mainly locate in the superior hospitals, such as some regional large and medium-sized hospitals. This situation leads these large and medium-sized hospitals to undertake most work of screening, diagnosis and treatment for the common eye diseases. The unbalanced condition is much more severe than other large departments such as internal medicine department and surgery department, and most of the primary hospitals are not even equipped with ophthalmologists. Therefore, the screening of common eye diseases has not been popularized due to the shortage of ophthalmologists in primary hospitals, and the resultant low coverage of community people. Eye disease screening with the aid of artificial intelligence appears to be a good solution.

But on one side, the hardware of the existing artificial intelligence aided eye disease screening system is high in cost and expertise requirement, and is more dependent on professional equipment and personnel in large and medium-sized hospitals. The artificial intelligence aided eye disease screening system, which can be applied to primary hospitals, also at least needs the participation of ophthalmic professionals to realize the disease screening function. Therefore, it is very difficult to introduce the existing artificial intelligence aided eye disease screening system to perform screening work in the primary hospitals in the situation that most primary hospitals lack the ophthalmic screening function and doctors with ophthalmic professional skills.

On the other hand, many common eye diseases can cause blindness including cataract, glaucoma, pathological myopia, age-related macular degeneration, and the like. However, the existing artificial intelligence aided screening system can only screen a single disease (e.g., diabetic retinopathy) which is inadequate for the improvement of the overall screening effect of the common blind-causing eye diseases.

### SUMMARY OF THE INVENTION

The invention aims to overcome at least one defect (deficiency) of the prior art, and provides an artificial intelligence eye disease screening service method and system, so as to keep a balance in the ophthalmic medical resources between primary hospitals and superior hospitals, and achieve a highly efficient eye disease screening with wide coverage.

The invention adopts a technical scheme as follows.

An artificial intelligence eye disease screening service method, includes: a step of acquiring ophthalmic examination data, including acquiring ophthalmic examination data of a user, wherein the ophthalmic examination data is obtained by a technician in a primary hospital through an ophthalmic examination on the user; a step of ophthalmic examination data screening, including performing an artificial intelligence screening on the ophthalmic examination data to obtain a screening result; and a step of generating ophthalmic examination result, including determining whether the screening result is normal or not, generating an ophthalmic examination report if the screening result is normal, and uploading the ophthalmic examination data to an ophthalmologist of a superior hospital for review and generating the ophthalmic examination report according to a review result if the screening result is abnormal.

Further, the step of ophthalmic examination data screening specifically adopts a corresponding artificial intelligence model for different kinds of eye diseases, and carries out the artificial intelligence screening on the ophthalmic examination data.

Further, the step of ophthalmic examination data screening specifically determines which type of the eye disease requires the artificial intelligence screening according to the type of the input ophthalmic examination data; and adopts an artificial intelligence model corresponding to the determined type of eye disease, and carries out the artificial intelligence screening on the ophthalmic examination data.

Further, the ophthalmic examination data includes one or more among visual acuity, fundus images, anterior segment images, eye pressure, and diopters.

Further, the method also includes the step of generating the ophthalmic examination report: acquiring personal information input by the user, in which the identity information and/or health information are included; and determining whether to generate the ophthalmic examination report according to whether the personal information meets a preset requirement or not, and sending the ophthalmic examination report to the user.

An artificial intelligence eye disease screening service system, includes: an ophthalmic examination data acquisition module, which is used for acquiring ophthalmic examination data of the user and sending the ophthalmic examination data to an ophthalmic artificial intelligence technology platform, in which the ophthalmic examination data is obtained by the technician in the primary hospital through the ophthalmic examination on the user; an ophthalmic artificial intelligence technology platform, which is used for carrying out the artificial intelligence screening on the ophthalmic examination data to obtain the screening result; and an ophthalmic examination result generation module, which is used tbr determining whether the screening result is normal, generating the ophthalmic examination report if the screening result is normal, and uploading the ophthalmic examination data to the ophthalmologist of the superior hospital for review and generating an ophthalmic examination report according to the review result if the screening result is abnormal.

Further, the ophthalmic artificial intelligence technology platform is particularly used to adopt the corresponding artificial intelligence models for different kinds of eye diseases, and performing the artificial intelligence screening on the ophthalmic examination data.

Further, the ophthalmic artificial intelligence technology platform includes: an type judging module of eye disease, which is used for determining which type of the eye disease requires the artificial intelligence screening according to the type of input ophthalmic examination data; and an artificial intelligence screening module, which is used for adopting the artificial intelligence model corresponding to the determined type of eye disease, and performing the artificial intelligence screening on the ophthalmic examination data.

Further, the system also includes an ophthalmic examination report generation module, which is used for acquiring the personal information input by the user, in which the identity information and/or health information are included, and determining whether to generate the ophthalmic examination report according to whether the personal information meets the preset requirement or not, and sending the ophthalmic examination report to the user.

Further, the system also includes an ophthalmic examination appointment module, which is used for acquiring appointment information of the user, and making an appointment with the technician in the primary hospital perform the ophthalmic examination on the user.

Compared with the prior art, the present invention has the following beneficial effects:

(1) The task of ophthalmic examination sink to the primary hospital, a unified artificial intelligence screening is performed, and the data having been screened out as abnormal are sent to the superior hospital again for review. The method fully considers the current situation that most primary hospitals have no professional ophthalmologist, and provide examination equipment which is lightweight, portable, highly efficient and easy to operate in the primary hospitals. The equipments can be operated by a technician after a simple training, and the technician do not need to provide diagnosis, treatment, or any interpretation of the examination report. Thereby the method can save the valuable medical resources of primary health care organizations make the screening of common blind-causing diseases available for more people at the basic level, ease the heavy burden of ophthalmic care in the superior hospitals, and balance the eye medical resources between the primary hospitals and the superior hospitals;

(2) The ophthalmic examination data obtained by the technician can be sent to the ophthalmic artificial intelligence technology platform for immediate artificial intelligence screening. The ophthalmic examination report can also be fed back to the user in time even if the user lives far away from the large and medium-sized hospitals. Thereby the ophthalmic screening can be timely and efficient;

(3) The artificial intelligence screening is performed through a ophthalmic artificial intelligence technology platform. And the abnormal screening result is uploaded to professional ophthalmologists of superior hospitals for review, which can establish an organic connection between the primary hospitals and the superior hospitals, rebuild people's trust in the ophthalmic services of basic level hospitals, and further ease the problem of unevenly-distributed medical resources;

(4) A plurality of artificial intelligence models are integrated. The artificial intelligence screening is performed on different eye diseases by adopting the corresponding artificial intelligence model, so that the user can obtain a multi-disease screening after the ophthalmic examination in a primary hospital. Therefore, the medical resources are integrated, and the medical cost is reduced;

(5) The artificial intelligence screening is performed on different eye diseases by adopting corresponding artificial intelligence model after determining the eye disease type on which the artificial intelligence screening needs to be performed in advance. Thereby the artificial intelligence screening can be more targeted, more efficient and more cost-effective;

(6) The user can make an appointment and be examined in the nearest primary hospital, and finally acquire an easy-to-understand report. The invention provides a one-stop eye disease screening service for the user.

### BRIEF DESCRIPTION OF THE DRAWINGS

FIG. 1 is a flow chart of the method of embodiment 1 in the present invention.

FIG. 2 is a composition diagram of the system of embodiment 2 in the present invention.

FIG. 3 is another composition diagram of the system of embodiment 2 in the present invention.

### DESCRIPTION OF EMBODIMENTS

The drawings are for illustrative purposes only and are not to be construed as limiting the invention. Some components in the drawings are omitted, enlarged, or reduced to better illustrate the following embodiments, and sizes of these components do not represent the sizes of actual products. It will be appreciated by those skilled in the art that some known structures and descriptions thereof may be omitted.

### Embodiment 1

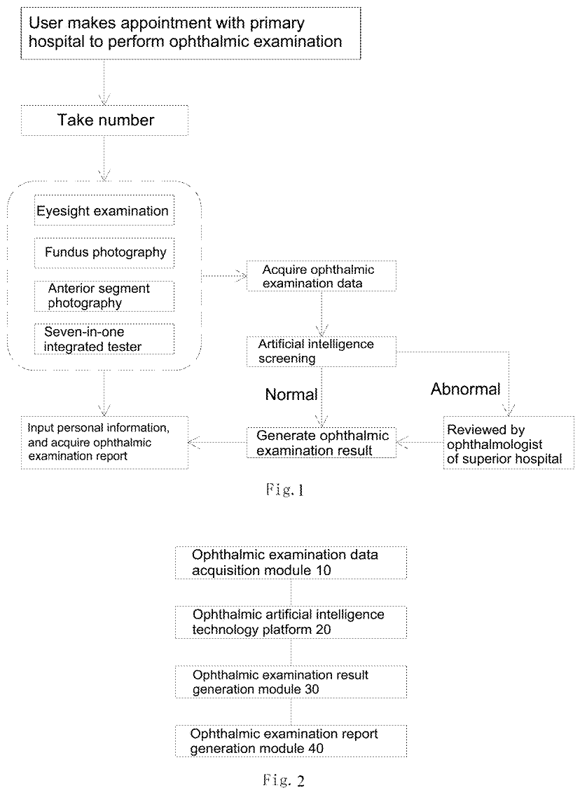

As shown in FIG. 1, the present embodiment provides an artificial intelligence eye disease screening service method, includes: a step of acquiring ophthalmic examination data, including acquiring ophthalmic examination data of a user, wherein the ophthalmic examination data is obtained by a technician in a primary hospital through an ophthalmic examination on the user; a step of ophthalmic examination data screening, including performing the artificial intelligence screening on the ophthalmic examination data to obtain a screening result; and a step of generating ophthalmic examination result, including determining whether the screening result is normal or not, generating an ophthalmic examination report if the screening result is normal, and uploading the ophthalmic examination data to an ophthalmologist of a superior hospital for review and generating the ophthalmic examination report according to the review if the screening result is abnormal.

The primary hospital refers to a medical institution with a relatively low grade, such as a township health center, a village clinic and a community hospital. The superior hospital is a medical institution with higher grade than the primary hospital, such as a regional large and medium-sized hospital.

Ophthalmic examination is performed by a trained technician in a primary hospital for the user. The ophthalmic examination data obtained is selected for the artificial intelligence screening, and in particular, the ophthalmic examination data can be uploaded to an ophthalmic artificial intelligence technology platform built by collaborative primary hospitals and superior hospitals in a unified manner to perform the artificial intelligence screening. An initial screening result can be obtained through the artificial intelligence screening. When the screening result is normal, an ophthalmic examination report can be generated directly. When the screening result is abnormal, the result can be uploaded to professional ophthalmologists in superior hospitals for review and further determining and/or correcting, and then the ophthalmic examination report is generated.

It can be understood that, when the screening result is normal, it means that the user is diagnosed with no eye disease through the artificial intelligence screening; when the screening result is abnormal, it means that the user is diagnosed with eye disease or suspected of having one through the artificial intelligence screening.

It can also be understood that, when the screening result is normal, it means that the user is diagnosed without or suspected of having no eye disease through the artificial intelligence screening; when the screening result is abnormal, it means that the user is diagnosed with or suspected of having eye disease through the artificial intelligence screening.

The task of ophthalmic examination sink to the primary hospitals, the obtained ophthalmic examination data is performed a unified artificial intelligence screening, and abnormal data are sent to the superior hospital again for review. The primary hospital only needs to be equipped with examination equipment which is lightweight, portable, highly efficient and easy to operate. The examinations can be operated by a simply trained technician, who does not need to provide diagnosis and treatment. Thereby the method can save the valuable medical resources at the basic level, make the screening of common blind-causing eye diseases available for more people, ease the burden of the ophthalmologists in the superior hospitals, and balance the eye medical resources between collaborative hospitals.

The ophthalmic examination data obtained by the technician in the primary hospital can be immediately and uniformly subjected to the artificial intelligence screening even if the user lives far away from the large and medium-sized hospitals, so that the ophthalmic screening can timely and highly efficient.

The abnormal screening result is uploaded to professional ophthalmologists of superior hospitals for review through the artificial intelligence technology platform, which can establish an organic connection between collaborative primary hospitals and superior hospitals, rebuild people's trust in the ophthalmic services at the basic level, and further ease the problem of unevenly-distributed medical resources.

In the present embodiment, the method also includes the step of generating the ophthalmic examination report: acquiring personal information input by the user, which includes identity information and/or health information; and determining whether to generate an examination report or not according to whether the personal information meets a preset requirement or not, and sending the examination report to the user.

The ophthalmic examination result can further generate an easy-to-understand report for the user. The user can acquire the report by inputting correct and satisfactory personal information. The method provides a one-stop eye disease screening service for the user. The personal information can include identity information and health information, in which the identity information can include the ID number or the medical card number of the user, and the health information can include the current health condition, medical history, and so on.

In the specific implementation, the user can input personal information through a mobile and/or a fixed user terminal, and the examination report can be sent to the user terminal for viewing.

In the present embodiment, the method also includes the step of ophthalmic examination appointment: acquiring the appointment information of the user, and making an appointment with technicians of collaborative primary hospitals to perform the ophthalmic examination on the user.

The user can make an appointment and take a number to be received by the technician in the primary hospital. Thereby the user is provided with an eye disease screening service in order at the appointment time.

In the specific implementation, the user can input appointment information through a mobile and/or a fixed user terminal.

In the present example, the ophthalmic examination data includes one or more among visual acuity, fundus images, anterior segment images, intraocular pressure, and refractive status.

In one of the embodiments, the step of ophthalmic examination data screening specifically adopts a corresponding artificial intelligence model for different kinds of eye diseases, and carries out the artificial intelligence screening on the ophthalmic examination data.

Different artificial intelligence models can be trained for different types of eye diseases, the artificial intelligence screening can be performed for different kinds of eye diseases adopting the corresponding artificial intelligence model after the ophthalmic examination data is acquired, so that the user can acquire a one-stop multi-disease screening after being performed ophthalmic examination in the primary hospital. Therefore the medical resources are highly integrated, and cost-effective.

In another implementation, the step of ophthalmic examination data screening specifically determines which type of the eye disease requires the artificial intelligence screening according to the obtained type of the ophthalmic examination data; and adopts an artificial intelligence model corresponding to the determined type of eye disease, and carries out the artificial intelligence screening on the ophthalmic examination data.

The screening of different kinds of eye diseases differs from each other to some degree in the requiring types of ophthalmic examinations. Determining the overall visual function needs eyesight test to acquire the visual acuity of the user. Common fundus diseases (e.g., diabetic retinopathy, age-related macular degeneration, etc.) require the fundus photography to acquire a fundus image. Common anterior segment diseases (e.g., age-related cataracts, etc.) require the anterior segment photography to acquire the anterior segment image. Glaucoma, myopia and the like require the examinations including intraocular pressure and refraction.

Different ophthalmic examinations can obtain different kinds of data, which is also corresponding to the screening of different kinds of eye diseases. Therefore which type of eye disease requires the artificial intelligence screening can be determined by the obtained types of ophthalmic examination data. For example, if the ophthalmic examination data includes visual acuity, intraocular pressure, refraction, it can be determined that the types of eye disease requiring the artificial intelligence screening are the common eye diseases such as glaucoma, myopia and the like. If the ophthalmic examination data includes the fundus image, then it can be determined that the types of eye diseases requiring the artificial intelligence screening are common fundus diseases. If the ophthalmic examination data includes the anterior segment image, then it can be determined that the types of eye diseases requiring the artificial intelligence screening are common anterior segment diseases.

The type of eye diseases requiring the artificial intelligence screening is determined in advance, then the artificial intelligence screening is performed on the type of eye disease by adopting the corresponding artificial intelligence model, so that the artificial intelligence screening can be more targeted, more efficient and more cost-effective.

In the specific implementation, the primary hospital is equipped with a fundus camera, a slit lamp anterior segment camera, and a seven-in-one integrated instrument and the like, which are to be operated by a technician to perform ophthalmic examination on the user. The seven-in-one instrument, among these devices, can carry out routine measurement on items including intraocular pressure, refraction and the like.

### Embodiment 2

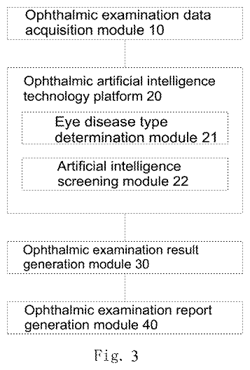

As shown in FIG. 2, the present embodiment provides an artificial intelligence eye disease screening service system, includes: an ophthalmic examination data acquisition module 10, which is used for acquiring ophthalmic examination data of the user and sending the ophthalmic examination data to an ophthalmic artificial intelligence technology platform 20, in which the ophthalmic examination data is obtained by the technician in the primary hospital through the ophthalmic examination on the user; an ophthalmic artificial intelligence technology platform 20, which is used for carrying out the artificial intelligence screening on the ophthalmic examination data to obtain the screening result; and an ophthalmic examination result generation module 30, which is used for determining whether the screening result is normal, generating the ophthalmic examination report if the screening result is normal, and uploading the ophthalmic examination data to the ophthalmologist of the superior hospital for review and generating an ophthalmic examination report according to the review if the screening result is abnormal.

The primary hospital refers to a medical institution with a relatively low grade, such as a township health center, a village clinic, and a community hospital. The superior hospital is a medical institution with the higher grade than the primary hospital, such as a regional large and medium-sized hospital.

Ophthalmic examination is performed by a trained technician in collaborative primary hospitals for the user. The ophthalmic examination data acquired by the ophthalmic examination data acquisition module 10 is selected and uploaded to the ophthalmic artificial intelligence technology platform 20 built by collaborative primary hospitals and the superior hospitals in a unified manner to perform the artificial intelligence screening. An initial screening result can be obtained through the artificial intelligence screening. Determining whether the screening result is normal or not through the examination result generation module 30. When the screening result is normal, an ophthalmic examination report can be generated directly. When the screening result is abnormal, the result can be uploaded to the professional ophthalmologists in the superior hospitals for review and further determining and/or correcting, and then the ophthalmic examination report is generated.

It can be understood that, when the screening result is normal, it means that the user is diagnosed with no eye disease through the artificial intelligence screening; when the screening result is abnormal, it means that the user is diagnosed with eye disease or suspected of having eye disease through the artificial intelligence screening.

It can also be understood that, when the screening result is normal, it means that the user is diagnosed without or suspected of having no eye disease through the artificial intelligence screening; when the screening result is abnormal, it means that the user is diagnosed with or suspected of having eye disease through the artificial intelligence screening.

The task of ophthalmic examination sink to the primary hospitals, the obtained ophthalmic examination data is performed a unified artificial intelligence screening, and the data having been screened out as abnormal are sent to the superior hospital again for review. The primary hospital only needs to be equipped with examination equipment which is lightweight, portable, highly efficient and easy to operate. The equipments can be operated by a simply trained technician who does not need to provide diagnosis and treatment. Thereby the method makes the eye disease screening available for more people at the basic level, eases the burden of the ophthalmologists in the superior hospitals, and balances the ophthalmic medical resources between the primary hospitals and the superior hospitals.

The ophthalmic examination data obtained by the technician in the primary hospital through the ophthalmic examination on the user can be sent to the ophthalmic artificial intelligence technology platform 20 for the artificial intelligence screening immediately and uniformly. The ophthalmic screening can be timely and highly efficient even if the user lives far away from the large and medium-sized hospitals.

The artificial intelligence screening is performed through the ophthalmic artificial intelligence technology platform 20, and the abnormal screening result is uploaded to the professional ophthalmologists of the superior hospital for review, which can establish an organic connection between the primary hospital and the superior hospital, rebuild people's trust in the ophthalmic services at the basic level, and further ease the problem of unevenly-distributed medical resources.

In the present embodiment, the system also includes an ophthalmic examination report generation module 40, which is used for acquiring the personal information input by the user, which includes identity information and/or health information, and determining whether to generate the ophthalmic examination report according to whether the personal information meets the preset requirement or not, and sending the ophthalmic examination report to the user.

Through the ophthalmic examination report generation module 40, the ophthalmic examination result can further generate an easy-to-understand report for the user who can acquire the report by inputting correct and satisfactory personal information, which provides a one-stop eye disease screening service for the user. The personal information can include identity information and health information, in which the identity information can include the ID number or the medical card number of the user, and the health information can include the current health condition, medical history, and so on.

In the specific implementation, the user can input personal information through a mobile and/or a fixed user terminal, which sends the personal information input by the user to the ophthalmic examination report generation module 40. The ophthalmic examination report generation module 40 generates the examination report and then sends the report to the user terminal for viewing.

In the present embodiment, the system also includes an ophthalmic examination appointment module, which is used for acquiring appointment information of the user, and making an appointment with the technician in the primary hospital to perform the ophthalmic examination on the user.

Through the ophthalmic examination appointment module, the user can make an appointment and take a number to be received by the technician in the primary hospital, and is provided with an eye disease screening service in order at the appointment time.

In the specific implementation, the user can input appointment information through a mobile and/or a fixed user terminal.

In one of the embodiments, the ophthalmic artificial intelligence technology platform 20 is specifically used for adopting a corresponding artificial intelligence model for different kinds of eye diseases, and performing the artificial intelligence screening on the ophthalmic examination data.

Different artificial intelligence models can be trained for different types of eye diseases, and the ophthalmic artificial intelligence technology platform 20 integrates these different artificial intelligence models, which is more convenient compared with the existing artificial intelligence aided eye disease screening system which can only screen a single disease. The ophthalmic examination data acquisition module 10 sends the ophthalmic examination data to the ophthalmic artificial intelligence technology platform 20, and then the ophthalmic artificial intelligence technology platform 20 can perform the artificial intelligence screening on different kinds of eye diseases by adopting the corresponding artificial intelligence model, so that the user can acquire a one-stop multi-disease screening after being examined in the primary hospital. Therefore, the medical resources are integrated, and the medical cost is reduced.

As shown in FIG. 3, in another embodiment, the ophthalmic artificial intelligence technology platform 20 includes: an eye disease type determination module 21, which is used for determining which type of the eye disease requires the artificial intelligence screening according to the type of the ophthalmic examination data obtained; and an artificial intelligence screening module 22, which is used for adopting the corresponding artificial intelligence model, and performing the artificial intelligence screening on the ophthalmic examination data.

The screening of different kinds of eye diseases differs from each other to some degree in the requiring ophthalmic examinations. Determining the overall visual function needs eyesight test to acquire the visual function of the user. Common fundus diseases (e.g., diabetic retinopathy, age-related macular degeneration, etc.) require the fundus photography to acquire a fundus image. Common anterior segment diseases (e.g., age-related cataracts, etc.) require the anterior segment photography to acquire the anterior segment image. Glaucoma, myopia and the like require the examinations such as intraocular pressure and refraction.

Different ophthalmic examinations can obtain different kinds of ophthalmic examination data, which is also corresponding to the screening of different kinds of eye diseases, so that the eye disease type determination module 21 can determine which type of eye disease requires the artificial intelligence screening according to the type of ophthalmic examination data obtained. For example, if the ophthalmic examination data includes visual function, intraocular pressure, refraction, the eye disease type determination module 21 can determine that the type of eye disease requiring the artificial intelligence screening is a common eye disease such as glaucoma, myopia and the like. If the ophthalmic examination data includes a fundus image, the eye disease type determination module 21 can determine that the type of eye disease requiring the artificial intelligence screening is a common fundus disease. If the ophthalmic examination data includes an anterior segment image, the eye disease type determination module 21 can determine that the type of eye disease requiring the artificial intelligence screening is a common anterior segment disease.

The eye disease type determination module 21 determines which type of eye diseases requires the artificial intelligence screening in advance, then the artificial intelligence screening module 22 performs the artificial intelligence screening by adopting the corresponding artificial intelligence model. Thereby the artificial intelligence screening can be more targeted, more efficient and more cost-effective.

In the specific implementation, the primary hospital is equipped with a fundus camera, a slit lamp anterior segment camera, and a seven-in-one integrated instrument and the like which are to be operated by a technician to perform ophthalmic examination on the user. The ophthalmic examination data acquisition module 10 is connected with the fundus camera, the slit lamp anterior segment camera, and the seven-in-one integrated instrument respectively to acquire the ophthalmic examination data. The seven-in-one instrument, among these devices, can carry out routine measurement on intraocular pressure, refraction and the like.

Obviously, the foregoing embodiments of the present invention are merely example for clear illustration of the technical scheme in the invention, and are not intended to limit the specific embodiments of the present invention. Any modification, equivalent substitution or improvement, and the like within the spirit and principle of the claims of the present invention should be included in the scope of claims of the present invention.

1. An artificial intelligence eye disease screening service method, comprising:
a step of acquiring ophthalmic examination data, including acquiring ophthalmic examination data of a user, wherein the ophthalmic examination data is obtained by a technician in a primary hospital through an ophthalmic examination on the user;
a step of ophthalmic examination data screening, including performing an artificial intelligence screening on the ophthalmic examination data to obtain a screening result; and
a step of generating ophthalmic examination result, including determining whether the screening result is normal or not, generating an ophthalmic examination report if the screening result is normal, and uploading the ophthalmic examination data to ophthalmologists of collaborative superior hospitals for review and generating the ophthalmic examination report according to a review if the screening result is abnormal.

2. The artificial intelligence eye disease screening service method according to claim 1, wherein the step of ophthalmic examination data screening specifically includes adopting a corresponding artificial intelligence model for targeted kinds of eye diseases, and carrying out the artificial intelligence screening on the ophthalmic examination data.

3. The artificial intelligence eye disease screening service method according to claim 1, wherein the step of ophthalmic examination data screening specifically includes:
determining which type of eye disease requires the artificial intelligence screening according to the type of the ophthalmic examination data obtained; and
adopting an artificial intelligence model corresponding to the determined type of eye disease, and carrying out the artificial intelligence screening on the ophthalmic examination data.

4. The artificial intelligence eye disease screening service system according to claim 1, wherein the ophthalmic examination data includes one or more among visual acuity, fundus images, anterior segment images, intraocular pressure, and diopters.

5. The artificial intelligence eye disease screening service method according to claim 1, wherein the method also includes a step of generating the ophthalmic examination report:
acquiring personal information input by the user, wherein the personal information includes identity information and/or health information; and
determining whether to generate an examination report or not according to whether the personal information meets a preset requirement or not, and sending the examination report to the user.

6. An artificial intelligence eye disease screening service system, comprising:
an ophthalmic examination data acquisition module, which is used for acquiring ophthalmic examination data of a user and sending the ophthalmic examination data to an ophthalmic artificial intelligence technology platform, in which the ophthalmic examination data is obtained by a technician in a primary hospital through an ophthalmic examination on the user;
an ophthalmic artificial intelligence technology platform, which is used for carrying out an artificial intelligence screening on the ophthalmic examination data to obtain a screening result; and
an ophthalmic examination report generation module, which is used for determining whether the screening result is normal, generating an ophthalmic examination report if the screening result is normal, and uploading the ophthalmic examination data to an ophthalmologist of a superior hospital for review and generating the ophthalmic examination report according to the review if the screening result is abnormal.

7. The artificial intelligence eye disease screening service system according to claim 6, wherein the ophthalmic artificial intelligence technology platform is particularly used to adopt corresponding artificial intelligence models for targeted kinds of eye diseases, and perform the artificial intelligence screening on the ophthalmic examination data.

8. The artificial intelligence eye disease screening service system according to claim 6, wherein the ophthalmic artificial intelligence technology platform includes:
an eye disease type judging module, which is used for determining which type of eye disease requires the artificial intelligence screening according to the type of the ophthalmic examination data obtained; and
an artificial intelligence screening module, which is used for adopting the artificial intelligence model corresponding to the determined type of eye disease, and performing the artificial intelligence screening on the ophthalmic examination data.

9. The artificial intelligence eye disease screening service system according to claim 6, further comprising an ophthalmic examination report generation module, which is used for acquiring personal information input by the user, in which the personal information includes identity information and/or health information, and determining whether to generate the ophthalmic examination report according to whether the personal information meets a preset requirement or not, and sending the ophthalmic examination report to the user.

10. The artificial intelligence eye disease screening service system according to claim 6, further comprising an ophthalmic examination appointment module, which is used for acquiring appointment information of the user, and making an appointment with the technician in primary hospitals to perform the ophthalmic examination on the user.
